(19) United States
(12) Patent Application Publication
(10) Pub. No.: US 2014/0262884 A1
(43) Pub. Date: Sep. 18, 2014

(54) MEDICINE STORAGE ARRANGEMENTS AND METHODS OF ASSEMBLY AND USE

(71) Applicant: APOTHECARY PRODUCTS, INC., Burnsville, MN (US)

(72) Inventors: Robert N. Priebe, Bloomington, MN (US); Terrance O. Noble, Burnsville, MN (US)

(73) Assignee: Apothecary Products, Inc., Burnsville, MN (US)

(21) Appl. No.: 13/827,733

(22) Filed: Mar. 14, 2013

(51) Int. Cl. A61B 19/02; A61J 1/03

(52) U.S. Cl. CPC A61B 19/026; A61J 1/03
USPC 206/365; 206/538; 206/364

(57) ABSTRACT

Medicine storage arrangement methods of assembly and use are described. The arrangements include a multi-chamber pill dispensing arrangement; and, a holder arrangement for an alternate medical dispenser, such a medicine dispensing pen or a syringe. Methods of assembly and use are described.

### FIELD OF THE DISCLOSURE

The invention relates generally to storage containers and in particular to storage containers for medicines. The storage containers described and shown include multi-day pill container/reminders in a housing that also includes a holder arrangement for securing other medical dispensing equipment such as dispenser pens or syringes. Example assemblies, and methods of assembly and use, are disclosed.

### BACKGROUND

For persons who need to take medicine regularly, the need for a pill container that can be easily carried, stored and used, and which is organized to avoid confusion over dose, is desirable. In addition, in many instances, such persons need to manage alternate dispensing arrangements to pills, for example: dispensing pens, syringes, bottles, inhalers, etc. It is desirable to provide storage containers which not only allow for management of daily pill regimen, but also rapid and convenient collection, storage and access for alternate (non-pill) medicine dispensing arrangements.

Herein, features, techniques, and methods for providing such arrangements are described, and examples are depicted.

### SUMMARY

According to the present disclosure medicine dose containment arrangements, features thereof and techniques for assembly and use of such arrangements, are described. In general, the medicine dose containment arrangements comprise a cover section and a bottom section. A first selected one of the cover section of the bottom section includes a plurality of individual pill containers therein. A second selected one of the cover section and bottom section includes a holder arrangement for a medicine dispenser arrangement. Typically the holder arrangement is for a non-pill medicine dispenser arrangement, such as: a medicine dispenser pen; one or more medicine dispenser syringes; an inhaler; a small bottle containing liquid; etc.

There is no specific requirement in an assembly component feature or technique that involve all of the detail described herein, in order to obtain some benefit according to the present disclosure.

### BRIEF DESCRIPTION OF THE DRAWINGS

FIG. 1 is a schematic perspective view of a first embodiment of a medicine storage arrangement in accord with the present disclosure.

FIG. 2 is a schematic perspective view of the arrangement of FIG. 1, depicted open and with an example medicine dispenser, in the form of a pen dispenser, contained therein.

FIG. 3 is a schematic top plan view of the open arrangement of FIG. 2.

FIG. 4 is a schematic cross-sectional view of the arrangement of FIGS. 2 and 3 shown used with a dispensing pen; the view of FIG. 4 being taken generally along line 4-4, FIG. 3.

FIG. 5 is a schematic, enlarged, fragmentary view of an identified portion of FIG. 4.

FIG. 6 is a schematic exploded perspective view of the arrangement of FIGS. 2 and 3 depicted with a dispensing pen.

FIG. 7 is a schematic top perspective view of a base section of the assembly of FIGS. 2, 3 and 6.

FIG. 8 is a schematic top plan view of the base section of FIG. 7.

FIG. 9 is a schematic cross-sectional view taken generally along line 9-9, FIG. 8.

FIG. 10 is a schematic, enlarged, fragmentary schematic view of an identified portion of FIG. 9.

FIG. 11 is a schematic perspective view of a cover section of the arrangement of FIGS. 2, 3 and 6.

FIG. 12 is a schematic top plan view of the cover section of FIG. 11.

FIG. 13 is an enlarged fragmentary schematic cross-sectional view taken generally along line 13-13, FIG. 12.

FIG. 14 is a schematic cross-sectional view taken generally along line 14-14, FIG. 12.

FIG. 15 is a fragmentary schematic cross-sectional view taken generally along line 15-15, FIG. 12.

FIG. 16 is an enlarged fragmentary schematic cross-sectional taken generally along line 16-16, FIG. 12.

FIG. 17 is a schematic perspective view of a multi-chamber pill container/reminder component of the arrangement of FIGS. 2, 3 and 6.

FIG. 18 is a schematic top plan view of a component of FIG. 17.

FIG. 19 is a schematic end elevational view of the component of FIGS. 17 and 18.

FIG. 20 is a schematic cross-sectional view taken generally along line 20-20, FIG. 18.

FIG. 21 is an enlarged fragmentary schematic view of a first identified portion of FIG. 20.

FIG. 22 is an enlarged fragmentary schematic view of a second identified portion of FIG. 20.

FIG. 23 is an enlarged fragmentary schematic view of a third portion of FIG. 20.

FIG. 24 is a schematic top perspective view of a first chamber cover component of the arrangement of FIGS. 2, 3 and 6.

FIG. 25 is a schematic top plan view of the first chamber cover component of FIG. 24.

FIG. 26 is a schematic tab end view of the first chamber cover component of FIG. 24.

FIG. 27 is a schematic cross-sectional view taken generally along line 27-27, FIG. 25.

FIG. 28 is an enlarged fragmentary schematic view of an identified portion of FIG. 27.

FIG. 29 is a schematic top perspective view of the second chamber cover component of the arrangement of FIGS. 2, 3 and 6.

FIG. 30 is a schematic top plan view of the second chamber cover component of FIG. 29.

FIG. 31 is a schematic tab end view of the component of FIGS. 29 and 30.

FIG. 32 is a schematic cross-sectional view taken generally along line 32-32, FIG. 30.

FIG. 33 is an enlarged fragmentary schematic cross-sectional view of an identified portion of FIG. 32.

FIG. 34 is a schematic perspective view of a closure actuator member of the assembly of FIGS. 2, 3 and 6.

FIG. 34A is a schematic plan view of the closure actuator member of FIG. 33.

FIG. 35 is an end view of the closure actuator member of FIG. 34.

FIG. 36 is a schematic side view of the member of FIG. 34.

FIG. 36A is a schematic back side view of the member of FIG. 34.

FIG. 37 is a schematic cross-sectional view taken generally along line 37-37, FIG. 36.

FIG. 38 is a schematic perspective view of a holder member of the arrangement of FIGS. 2, 3 and 6.

FIG. 39 is a schematic plan view of the holder member of FIG. 38.

FIG. 40 is a schematic side elevational view of the holder member of FIGS. 38 and 39.

FIG. 41 is a schematic end view of the holder member of FIGS. 38-40.

FIG. 42 is a schematic cross-sectional view taken generally along line 42-42, FIG. 40.

FIG. 43 is a schematic closed perspective view of a second example medicine storage container arrangement to the present disclosure.

FIG. 44 is a schematic perspective view of the medicine storage container arrangement of FIG. 43, depicted open and shown used with two syringes stored therein.

FIG. 45 is a schematic top plan view of the arrangement in FIG. 44.

FIG. 46 is a schematic cross-sectional view taken generally along line 46-46, FIG. 45.

FIG. 47 is an enlarged fragmentary schematic cross-sectional view of an identified portion of FIG. 46.

FIG. 48 is a fragmentary cross-sectional view taken generally along line 48-48, FIG. 45.

FIG. 49 is an enlarged fragmentary schematic cross-sectional view of an identified portion of FIG. 48.

FIG. 50 is a schematic exploded perspective view of the assembly of FIG. 44 depicted also showing two syringes.

FIG. 51 is a schematic top perspective view of a bottom section of the assembly of FIGS. 44 and 50.

FIG. 52 is a schematic top plan view of the bottom section of FIG. 51.

FIG. 53 is a schematic cross-sectional view taken generally along line 53-53, FIG. 52.

FIG. 54 is a schematic, enlarged, fragmentary view of an identified portion of FIG. 53.

FIG. 55 is a schematic perspective view of a cover section of the assembly of FIGS. 44 and 50.

FIG. 56 is a schematic plan view of the cover section of FIG. 55.

FIG. 57 is a schematic cross-sectional view taken generally along line 57-57, FIG. 56.

FIG. 58 is a schematic cross-sectional view taken along generally line 58-58, FIG. 56.

FIG. 59 is an enlarged fragmentary schematic cross-sectional view taken generally along line 59-59, FIG. 56.

FIG. 60 is an enlarged fragmentary schematic cross-sectional view taken generally along lines 60-60, FIG. 56.

FIG. 61 is a schematic enlarged fragmentary view of an identified portion of FIG. 60.

FIG. 62 is a schematic perspective view of a multi-chamber pill storage/reminder component of the assembly of FIGS. 44 and 50, depicted with all chambers open.

FIG. 63 is a schematic top plan view of the component of FIG. 62.

FIG. 64 is a schematic end view of the component of FIG. 63.

FIG. 65 is a schematic cross-sectional view taken generally along line 65-65, FIG. 63.

FIG. 66 is an enlarged fragmentary schematic view of a first identified portion of FIG. 65.

FIG. 67 is an enlarged fragmentary schematic view of a second identified portion of FIG. 65.

FIG. 68 is an enlarged fragmentary schematic view of a third identified portion of FIG. 65.

FIG. 69 is a schematic perspective view of an internal hinged syringe cover component of the assembly of FIGS. 44 and 50.

FIG. 70 is a schematic plan view of the component of FIG. 69.

FIG. 71 is a schematic cross-sectional view of taken generally along line 71-71, FIG. 70.

FIG. 72 is an enlarged schematic fragmentary view of a first identified portion of FIG. 71.

FIG. 73 is an enlarged fragmentary schematic view of a second identified portion of FIG. 71.

FIG. 74 is a schematic perspective view of a closure actuator member of the assembly of FIGS. 44 and 50.

FIG. 75 is a schematic plan view of the closure actuator member of FIG. 74.

FIG. 76 is a schematic end view of the component of FIG. 75.

FIG. 77 is a schematic bottom view of the component of FIG. 75.

FIG. 78 is a schematic cross-sectional view taken generally along line 78-78, FIG. 77.

FIG. 79 is a schematic back side plan view of the component of FIG. 74.

### DETAILED DESCRIPTION

### I. Further Background

Medicines are taken by patients in a variety of forms; for example as: pill(s); oral liquid(s); and/or through use of inhaler(s); medical dispensing pen(s); and/or syringe injection(s). It is desirable to provide, in a single convenient storage and carrier assembly, secure storage of a variety of medicines that may be taken in alternate ways. For example, a diabetic may have a daily pill regimen as well as need for a medicine dispensing pen and/or a medicine dispensing syringe. It would be advantageous if the daily pill regimen and alternate dispenser (pen and/or syringe) were conveniently contained in an easy to fill, access and carry assembly.

Herein, arrangements that are convenient to manufacture, assemble, charge and use to accomplish the above are described. It is noted that the arrangements can be used in a variety of alternate applications with respect to medicine storage and access, as will be apparent from the following descriptions. It is also noted that the arrangements depicted in the figures, are shown in examples of use with various non-pill dispensers associated therewith. In one example, a dispensing pen is shown, and in another, two syringes are shown. The non-pill dispensers are not portions of the assembly, but are depicted to show examples of potential use.

There is no specific requirement that an assembly component, technique or feature include all of the detail described and shown in connection with the following examples, in order to obtain some benefit in accord with the present disclosure.

### II. A First Example Embodiment, FIGS. 1-42

### A. General Assembly Features and Use

The reference numeral 1, FIG. 1, indicates a medicine storage and use assembly or arrangement according to the present disclosure. The medicine storage and use arrangement 1 is configured with internal features to provide for the following: multi-day pill dispenser/reminder; and, holder arrangement(s) for an alternate (typically non-pill) medicine dispensing system. In a specific example disclosed in FIGS. 1-42, the holder arrangement is configured to hold a dispenser pen, for example a diabetic insulin pen.

In general, it is anticipated that the assembly 1 would be typically made of plastic although alternate materials are useable. Many of the features are configured in forms that can be conveniently manufactured with plastic molding techniques, and then can be conveniently assembled.

Although the features can be implemented in an arrangement of a variety of sizes, it is anticipated that a typical application will involve a closed assembly 1 having a longest dimension or length (L) of about 15-25 cm, a width (W) of about 4-8 cm, and a height (H) of about 2.5-7 cm.

Still referring to FIG. 1, assembly 1 includes an outer housing 2 having a top (second) section 3 and a bottom (first) section 4. The top (second) section 3 and bottom (first) section 4 are generally attached to one another along one side by a hinge not viewable in FIG. 1, to provide a housing 2 openable in a clamshell manner.

Attention is now directed to FIG. 2, in which the assembly 1 is depicted with the housing 2 open. Here, the hinge 6 is viewable extending along the direction of length. For the particular assembly 1 depicted, components of the assembly 1 can be formed from plastic as indicated above. Typically, however, within the hinge 6 is provided a hinge pin 7 of metal, for example, chrome steel. In many applications, this will be the only metal component of the assembly 1.

Referring to FIG. 2, it is noted that the assembly 1 is depicted in an example involving use with a medicine dispensing pen 10 therein. This is to indicate an example of use. The medicine dispensing pen 10 is a separate structure from the assembly 1, and represents a non-pill dispenser usable with the assembly 1.

Referring to FIG. 2, the assembly 1 includes: an internal multi-day pill storage and dispensing arrangement or component 12. In the example depicted, the pill storage and dispensing component 12 is configured with seven separate compartments 13, and thus is a seven day or one week pill dispenser/reminder. It is noted that alternate configurations with an alternate number of compartments 13 can be used in assemblies in accord with the techniques described herein.

The particular pill storage and dispensing assembly 12 depicted, is of a type sometimes characterized as a “pill reminder.” This is because the individual compartments 13 have separate labeled covers 13x thereon, with indicia for different days of the week; the indicia being shown generally at 14. In the example assembly 1 depicted, the indicia 14 includes braille or touch indicators shown at 15. Thus, for the example assembly 1, the covers 13x serve as reminders to the pill taker, based on the days of the week.

With respect to pill dispensing, operation of the assembly 12 is in a typical manner for many multi-day dispensers. Prior to the beginning of the seven day period, the individual chambers 13 will be charged with appropriate pills for that day. On the identified day, the user (or caregiver) would open the chamber for the identified day for access to the pills therein. The assembly provides a reminder to take the pills for each day; and, it provides for a reminder, after the chamber is empty, that the pills have in fact been taken or none are required. Of course, in alternate assemblies, the pills can be separated into individual chambers and compartments for different periods of the day.

It is noted that the assembly 1 is advantageous, for containing an array 12 of individually closed pill chambers 13, within a closeable clamshell housing 2. An advantage from this is that it tends to inhibit moisture from reaching an interior of the chambers 13, in which the pills are stored.

For the example arrangement 1 depicted, the multi-chamber pill storage and dispensing array or component 12 is a single component that is separately made from plastic and snap-fit in place in shell section 4. This will be typical and preferred, but is not required in all applications of the techniques described herein. The covers 13x, for the example depicted, are preferably secured by living hinges formed in a plastic, although alternatives are possible. The covers 13x are provided with front tabs 13z for convenient opening, and can be provided with a snap-fit closure arrangement as discussed below.

Typically, the arrangement 1 is configured with a one-way snap-fit engagement arrangement between the pill storage and dispensing arrangement 12 and the associated housing section 4. With such an arrangement, once the arrangement 12 has been snap-fit in place, it is not readily or easily removed. Configuration and features to provide for this are depicted in the various drawings.

Still referring to FIG. 2, the assembly 1 includes in a separate housing section from the pill dispenser 12, in this instance provided by (second) cover section 3. In cover section 3, an alternate medicine dispenser retainment or holder arrangement 17 is provided. The medical dispenser holder arrangement 17 is configured to secure in place, in a convenient manner, a dispenser, for medicine, other than the arrangement 12. Typically, the holder arrangement 17 is to secure a dispenser for medicine that is typically other than pills. In the example depicted, the medicine dispenser holder arrangement 17 is a holder arrangement 17 for a medicine dispensing pen 10. That is, the holder arrangement 18 is configured to retain, securely in place, an insulin dispensing pen 10x for a diabetic, or another type of dispensing pen.

For the particular assembly 1 depicted in the housing section 3 that contains the holder arrangement 17 is optionally provided at least one, and in the example a plurality of, (for example two) containers, chambers or compartments 20, 21. Each chamber, compartment or container 20, 21 is an independently openable/closeable compartment. In the example: one (compartment 20) is configured to be used for alternate pen tips; and, the other compartment (21) is configured for used pen tips. The optional chambers or compartments 20, 21 are depicted provided with closure cover arrangements, covers or lids 23, 24 respectively, configured for easy opening and closing, as discussed in more detail below.

From the above general descriptions, advantages and conveniences provided by the assembly 1 can be readily understood. The assembly 1 comprises a convenient storage and carrying compartment or housing 2 that is readily opened and, when opened, provides convenient access to (daily) pills and to alternate medicine dispenser(s), such as medical dispensing pen(s). The particular assembly depicted not only provides for convenient access to the dispensing pen(s), but includes conveniently located storage compartments for auxiliary equipment such as additional pen tips and/or for storage of used pen tips.

Of course, the holder arrangement 17 could be configured to hold alternate dispensing arrangements, such as syringes, discussed in an example below in connection with other Figs. and/or still alternate arrangements such as: inhalers; small bottles of liquid medicine, or even tubes of topical applicant. The cover section 3, in these latter instances, could be configured differently, for example it could have an alternately shaped holder and it would not necessarily have compartments corresponding to compartments 20, 21. These and other alternate ways of structuring the internal construction of the assembly 1 will be apparent, not only from the detailed descriptions herein of the present embodiment of FIG. 2, but also from an understanding of the variations represented by the alternate embodiment of FIGS. 43-79.

In FIG. 3, a top plan view of the assembly 1 is provided. Individual features already identified and described that are viewable include container 2, comprising sections 3 and 4. Section 4 includes the multi-day pill container arrangement 12 comprising individual compartments 13. Section 3 includes the holder arrangement 17, in the example, a holder arrangement 18 for a dispensing pen 10. Also contained within section 3 are the separate, openable and recloseable, chambers or compartments 20, 21. The two sections 3 and 4 are secured to one another along hinge 6.

In FIG. 4, a schematic cross-sectional view taken along line 4-4, FIG. 3 is provided. In FIG. 5, an enlarged fragmentary view of an identified portion of FIG. 4 is shown. Here an interlock arrangement or catch 13q between a cover 13x and a remainder 13r of a chamber 13 is shown.

### B. Components and Assembly of the Medicine Storage Arrangement 1

In the previous section, general features of the arrangement 1 were described. In this section, components usable to form the assembly 1 and which are configured for particularly convenient manufacture, assembly and use are described.

Attention is directed to FIG. 6, a schematic perspective exploded view of the arrangement 1. It is noted that in FIG. 6, the pen 10 is also depicted. Disregarding the pen 10 for a moment, since it is not part of the assembly 1, the remaining portions of FIG. 6 generally depict individual components that would be preformed, and from which the assembly 1 can be readily assembled. Referring to FIG. 6 (and disregarding the pen 10) for the assembly 1, the only component that would not typically be formed from plastic, is hinge pin 17, which would typically be formed from metal such as a polished chrome steel pin.

Still referring to FIG. 6, a listing of the individual components is as follows: clamshell first section or base 26 (which will form section 4); clamshell second section or cover 27 (which will form section 3); multi-day pill container 28 (which will form assembly 12); first storage cover 29; second storage cover 30; push panel or latch actuator member 31; and, holder member 17.

As indicated above, the various members cataloged above, can be separately made and then readily assembled together to form the assembly 1. In remaining portions of this section, convenient configurations for accomplishing this are described and shown.

Attention is now directed to FIGS. 7-10, in which features of the base or bottom (first) section 26 are described and shown. This section 26, is an example section that can be used as section 4, FIGS. 1 and 2. Referring to FIG. 7, the base section 26 is shown in perspective view. The base section 26 includes a bottom cover 37 and a perimeter wall 38. The perimeter wall 38 includes a back wall section 39b with hollow tube hinge sections 39t thereon. Opposite back wall 39b is front wall 39f. Also, wall 38 includes side/end sections 38e.

In FIG. 8, a plan view of section 26 is depicted. In. FIG. 9, a schematic cross-sectional view is shown. In FIG. 10 an enlarged cross-sectional view of an identified portion of FIG. 9 is shown.

In normal use of the resulting assembly 1, front wall 39f would face the user, and back wall 39b would extend away from the user.

It is expected that a base section 26 of the type depicted in FIGS. 7-10 can be readily configured to be molded from a plastic such as an FDA approved ABS. Selected features depicted allow for attachment of components as will be apparent from further discussion below. The section 26 is specifically configured for convenient attachment thereto of the pill dispenser/reminder assembly 28; and, attachment of the latch actuator member or arrangement 31 for use in closing/opening the resulting arrangement 1.

Attention is now directed to FIGS. 11-16, in which features of the cover (second) section 27 are depicted. The cover section 27 is usable to form housing section 3, FIGS. 1 and 2.

In FIG. 11, the cover section 27 is shown in schematic perspective view. It includes a top 43 and perimeter wall or rim 44. The perimeter rim 44 includes a back wall 45b with tubular hinge sections 45t thereon that will align with sections 39t, FIG. 7, when the clamshell housing 2 is formed, for receipt of the hinge pin 25. Opposite back wall 45b is front wall 45f. The rim 44 also includes side/end walls 44e.

When assembly 1 is open for use as shown at FIG. 2, typically the back wall 45b is oriented adjacent back wall 39b, and thus wall section 45b actually faces the user. Front wall section 45f, then, would normally face away from the viewer, when the assembly is open as shown in FIG. 2. However, when the assembly 1 is closed, as shown in FIG. 1, but oriented toward the user for use, front wall section 45f would face the user and back wall section 45b would face away from the user.

Section 43, FIG. 11, actually forms a bottom of section 27 when the assembly is open as shown in FIG. 2. However, when the assembly 1 is closed as shown in FIG. 1, section 43 is the top of the assembly 1.

The particular cover section 27 depicted, includes internal compartment wall arrangements 47, 48, oriented to define walls of chambers 20 and 21 respectively in assembly 1. The particular configuration for section 27 depicted, is configured to be readily moldable from a plastic such as an FDA approved ABS.

Attention is now directed to FIG. 12, a top plan view of section 27. At 50, attachment or locator stakes or pins are shown formed integral with the top 43, to provide for attachment of the holder 17, as discussed below.

In FIG. 13, a cross-sectional view taken along line 13-13, FIG. 12 is viewed. In FIG. 14, a cross-sectional view taken along line 14-14, FIG. 12 is viewable. In FIG. 15, a schematic cross-sectional view taken along line 15-15, FIG. 12 is viewable. In FIG. 16, an enlarged fragmentary cross-sectional view taken along line 16-16, FIG. 12 is viewable.

Selected features viewable in FIGS. 12-15 provide for convenient assembly as will be understood from discussion below.

Attention is now directed to FIGS. 17-23, in which the pill multi-chamber pill dispensing arrangement or array 28, useable as arrangement 12, FIG. 2 is depicted. In FIG. 17, the pill dispensing arrangement 28 is shown in top perspective view with all of the individual chambers 13 fully open. It is expected that the component or arrangement 28 would typically be formed (molded) as a single integral construction, from a plastic such as an FDA approved random co-polymer polypropylene (pp). Such a material will provide desirable flexibility for operation of the living hinges 13h, and also sufficient flexibility for a portion described below that relate to snap-fit assembly. The individual covers 13x would be typically connected to a remainder of arrangement 28 by the living hinges 13h, and would be separately pivotable to cover associated, individual, ones of the compartments 13.

In FIG. 18, the pill dispenser 28 is shown in plan view, with chambers 13 open. In FIG. 19, an end view is shown, again with chambers 13 open. In FIG. 20, a cross-sectional view taken along line 20-20, FIG. 18, is shown.

Referring to FIGS. 19 and 20, each chamber 13 can be viewed as having a front wall 13f, a rear wall 13r and a bottom 13b. The front 13f generally joins the bottom 13b with an internal curved or ramped configuration as shown at 13n, FIG. 19. The ramp 13n facilitates removal of pills resting on base 13b, by scooping or pulling them toward the user, when the assembly is used in the orientation shown in FIG. 2, and with the user facing the configuration with the writing or words upright.

The lower chamber 13c of the compartment 13, is generally formed by the front and rear walls 13f, 13r as shown, and also opposite sidewalls 13s, 13t, FIG. 18. The chamber 13c as a result has an upper edge 13e, FIG. 19, over which the closure 13x fits, during closure of the selected chamber 13c.

In FIG. 21, an enlarged fragmentary view of an identified portion of front wall 13f is provided. Adjacent the upper or open edge 13e and recessed downwardly therefrom along an interior surface 13i, the front wall 13f includes a first member 53 of a snap-fit closure arrangement to facilitate maintaining the associated cover 13x closed when intended, as discussed below.

In FIG. 23, a portion of cover 13x is depicted, the portion being as being indicated in FIG. 20. The cover 13x includes thereon a rim extension 54 configured to engage upper edge 13e, FIG. 19, during closure. The rim extension 54 for the particular assembly depicted, is configured to project into an interior of chamber 13c, to be surrounded by upper edge 13e, during closure. Alternates are possible.

Referring to FIG. 23, section 54f of rim 54 is positioned to fit along interior section 13i of front edge 13f, during closure. Section 54f includes a second (flexible) member 55 of a snap-fit closure arrangement 56. The second member 55 is configured to engage the first member 53 in a releasable snap-fit manner, in order to maintain the cover 13x closed, until it is desired that it be opened. The closure arrangement 56 for the assembly shown, comprises a recess 53 in the sidewall 13f and a flexible projection member 55 in the cover 13x. The flexible member 55 can then engage the recess 53 in a friction lock. Engagement during closure was referenced above along front 1f, FIG. 5.

In FIG. 22, an enlarged fragmentary view along hinge portion 13h is shown. From inspection of FIGS. 20 and 22, it can be understood that the depicted hinge 13h is a living hinge, in the material of the compartment 13c, for the example depicted.

Still in reference to the pill dispensing arrangement 28 of FIGS. 17-23, attention is directed to features that allow for convenient installation and retainment of arrangement 28 within base section 26. In particular, and referring to FIG. 18, a flexible peripheral flange 60 is viewable. Peripheral flange 60 is provided with end sections 60e that are sized, and are sufficiently flexible, to snap-fit under opposite retainer projections 26p, FIGS. 7 and 8, in base section 26. Further, the flange 60 includes front section 60f that is sized and configured (and is sufficiently flexible) to snap-fit underneath front retainer projections 38p in cover section 26, FIG. 8. Finally, referring to FIGS. 8 and 9, section 26 is provided with spacers 37f therein, which will be engaged along rear wall 13r, FIG. 19, of pill container 28, helping to keep the pill container forwardly biased in a secure snap-fit arrangement.

In general, then, assembly of the pill section 28 to the housing section 26 can be a matter of simple snap-fit to secure engagement. It is noted that typically the snap-fit engagement between the container component 28 and the housing base 26 will be such that disconnection in inhibited to a reasonable extent. That is, preferably a snap-fit is oriented to be one way so that engagement is relatively simple, but disengagement is somewhat more difficult. Such a one-way snap-fit is provided by shaping projections 26p, 38p appropriately. In general, this is accomplished by having upper surfaces of the projections 26p, 38p and provide for a smooth transition to the snap-fit, but with lower surfaces, which engage the flange 60 after snap-fit occurs, providing for a sharp angle and less ability to disconnect.

Attention is now directed to an assembly of selected features of the cover section 3, FIG. 2. In particular, attention is directed to the holder member of arrangement 17, which is provided by pen holder member 18. The holder member 18 for the example depicted, is shown and described in FIGS. 38-42.

In FIG. 38, the pen holder member 18 is shown in perspective view. The example pen holder member 18 is configured so that it can be formed as a single integral member, for example, from an FDA approved pom/acetal. The example holder member 18 depicted includes a central spine or section 70 with spaced members 71 configured for secure engagement with an intended medical dispersion member, in this instance, the dispensing pen. The example holder member 18 depicted is a c-clip arrangement and, the members 71 are spaced c-clips, although alternatives are possible.

In FIG. 39, a top plan view of the holder member 18 is shown. At 73, mounting apertures are shown positioned to engage stake ends 50, FIG. 12, in the cover. The holder member 18, then, can be positioned in place by being pushed over the stakes 50, with individual stakes 50 projecting through individual ones of the apertures 73. Heat staking, sonic welding, adhesive or other techniques can be used to secure the parts 18/27 together, preferably such that disconnection is not readily achieved.

Referring to FIG. 41, an end view, each of the members 71, comprises a c-clip with flexible side members 71s configured for snap-fit engagement with the appropriate medical dispenser, in this instance, a pen. In FIG. 42, a schematic cross-sectional view taken along line 42-42 is shown. In FIG. 40, a side elevational view is shown.

It is noted that a variety of alternate holder arrangements can be used instead of the c-clip holder arrangement. However, c-clip holder arrangement will be particularly convenient, when the medical dispenser is a pen, or has a similar cross-section so that the dispenser can be very easily pushed in place and removed, repeatedly.

In FIGS. 24-28, a lid, cover or cover member 29 for one of the compartments 20, FIG. 2 is shown. In FIG. 24 a perspective view is provided, and in FIG. 25 the cover member 29 is viewable in top plan view. The cover member 29 is configured for a press, snap-fit, engagement in association with the compartment 20, FIG. 6, in opening/closing association with sidewall 17, FIG. 11. The cover section 29 depicted is preferably configured to provide a hinge attachment, so that it can be repeatedly opened and closed without removal from the assembly 1. In FIG. 24, the cover 29 is depicted with a cover section 29s, a hinge section 29h, and an attachment tab or mounting projection 29t. In FIG. 25, a top plan view of section 29 is depicted. In FIG. 26, a closure end view is shown, with flexible friction closure tab 29x viewable. In FIG. 27, a cross-sectional view taken along line 27-27, FIG. 25 is shown. In FIG. 28, an enlarged fragmentary view of an identified portion of FIG. 27 is provided.

Referring to FIGS. 25 and 27, end wall, tab or mounting projection 29t is shown with a snap-fit or friction fit projection member 29p thereon configured for convenient one way press and snap-fit. In particular, tab 29t sized and configured to be pushed into a corresponding recess 80 in section 26, FIG. 6, for snap-fit and retaining engagement between projection 29p and receiver 80r. Such a recess would be positioned on end wall 84. In opposite wall 85, a corresponding receiver 81 (to receiver 80r) in end wall 85 is provided for releasable engagement with an analogous potion of another cover, for snap-fit engagement.

Referring to FIG. 12, compartment 20, which is formed by container wall 47, can be viewed as having first and second, opposite, long walls 47a, 47b; and, first and second, opposite, end walls 47c, 47d. Between wall 47c and end wall 84, is provided recess or receiver space (mounting projection receiving slot) 80. The receiver space 80 is aligned with recess 80r, FIG. 6. The receiver space 80 is sized, to snuggly receive, projecting therein, retaining tab (hinge projection) 29t on cover 29. Thus, during assembly, retaining tab (hinge projection) 29t is pushed into the recess (or hinge projection receiving slot) 80 until projection 29p engages recess 80r and lock fit occurs. Preferably the snap-fit is configured one way, as shown, so that when assembly occurs, disengagement is difficult.

Referring now to FIGS. 26-28, attention is directed to closure tab 29x, which is a flexible tab having a first member 89 of a snap-fit closure arrangement thereon. The flexible tab 29x is sized and configured to be received, in a snap-fit manner, in a recess in association with chamber 47. The snap-fit closure will be by analogy to a snap-fit engagement described below in connection with cover 60.

In FIGS. 29-33, an analogous cover 60, to cover 29, but constructed for section 48 is shown. Cover 60 can be identical to cover 29, except for orientation of the indicia thereon. Like features are indicated with the same reference numerals, as a result.

Referring to FIG. 32, attention is directed to a flexible closure tab 29x. The flexible closure tab 29x is shown in enlarged view, in FIG. 33. It is sized to snap-fit engage a recess, in the associated chamber, when used. In FIG. 11, an example of such recess is shown at 61r. In this manner, lid cover or cover member 60 is retained closed. A similar closure retainer, as indicated above, can be used with chamber 20.

Referring to FIG. 6, the final component of assembly 1 not previously described, is the latch actuator member 31. Latch actuator member 31 is viewable in FIGS. 34-37.

The latch actuator member 31 comprises, in the preferred arrangement depicted, a single push panel or integral molded latch piece 140 as viewable in FIG. 34. It is configured to be mounted on the bottom section 26, and to releasably engage a portion of the top section 27, when the top section 27 is closed over the bottom section 26.

For mounting of the latch piece 140, the bottom section 26 is configured as shown in FIGS. 7 and 8. In particular, the front wall 39f, FIG. 7, includes a central recess section 38r therein, over which the latch member 140, FIG. 6, will fit when mounted. Referring to FIG. 8, the recess section 38r includes a central recessed wall section 38w, and opposite end sections 38e. Positioned on the opposite wall sections 38e are pivot posts 141 for pivotal mounting of the latch member 140. In addition, the rear wall 38w includes an upper edge 38u with a pair of projections 38p thereon, to facilitate closure.

Turning now to the latch member 140, FIGS. 34-37, again it is configured in the particular preferred arrangement depicted, so that it can be molded from a plastic such as an FDA approved pom/acetal, as a single piece. In FIG. 34, a perspective view is provided, facing the front and top portion of the latch member 140. The latch member 140 can be viewed as having opposite ends 140e. One of the ends 140e is viewable in FIG. 34, as having a pivot mounting receiver 140r thereon, for pivoting engagement with one of the posts 141, FIG. 8. The opposite end would generally be a mirror image and also be configured for engaging one of the posts 141.

In FIG. 35, an end view directed toward one of the ends 140e is shown. The recess 140r can be seen to have a narrow neck section 140n, so that once pushed over a post 141 it will be retained in place.

As latch member 140 is pivotally mounted on the bottom section 26, FIGS. 1, 2 and 7, through rotational engagement with the posts 141 on the bottom section 26, a lower portion 140x of the latch 140 can pivot toward and away from the bottom section 4, 26, over a small arc of rotation. As it does this, post or projection arrangement 150, FIG. 34, which will generally project underneath projections 38p, FIG. 8, can pivot to bias a portion of the cover 3, 27. In particular, and referring to FIG. 9, each of the projections 38p is configured as a hook, over a lower recess 151. The recess 151 is more readily viewable in the enlarged fragmentary view of FIG. 10.

The access cover 27, FIG. 12 is provided with a front wall 45f having a pair of hook projections 155 adjacent thereto. The hook projections 155 are configured to respectively engage, in a hook manner, projections 38p in the bottom section 26, when the cover 27 is closed over the base 26. This will lock the container 2 closed. To open the container, one presses on the lower portion 140x of the latch member 31, which will pivot the latch member 31 on the pivot mounts 141, pushing the projections 150 upwardly and forwardly. This will push the hooks 155 out from engagement underneath the hooks 38p, releasing the cover 27 for pivoting around the hinge 6.

Of course a variety of alternate latch and latch actuator arrangements can be used. The particular arrangement depicted is convenient, as it does not provide for substantial additional increase and profile of the assembly 1, it can be conveniently operated on a push basis to open and simple to closure to snap-fit close; and, it can be configured from plastic materials.

### III. An Alternate Embodiment, FIGS. 43-79

### A. General Features, FIG. 43-50

As explained above, in general terms medicine container arrangements according to the present disclosure, comprise housings configured to contain, in an advantageous manner, multi-chamber pill dispensers and, at the same time, other medicine dispensers. In the embodiment described previously in connection with FIGS. 1-42, the assembly 1 depicted was particularly well configured to include, in addition to a multi-day pill dispenser, a dispenser pen 10.

In some instances, the alternate dispenser to be securely contained within the assembly will be one or more syringes. An example assembly applying the principles of the present disclosure in the context of such an arrangement, can be understood from the assembly of use of FIGS. 43-50 and the piece part views of FIGS. 51-79. In this section, overall assembly features will be discussed and summarized, in connection with FIGS. 43-50.

In FIG. 43, a second assembly 201 in accord with the present disclosure is depicted. The assembly 201 generally comprises a housing 202 having a top (second) section 203 and a bottom (first) section 204. For the particular assembly 201 depicted, as with assembly 1, the two sections 203, 204 are joined to one another to form a housing 2 that opens in clamshell member, by pivoting around hinge 206, FIG. 44.

In FIG. 44, the assembly 201 is depicted open, i.e. with top (second) section 203 having been pivoted around hinge 206 away from closure of the bottom (first) section 204.

The bottom section 204 can be viewed as having a multi-chamber pill dispenser assembly 212 therein. In some applications, the bottom section 204 can be identical to the bottom section 4, FIG. 2, and the dispenser 212 can be identical to dispenser 12, and can be configured and secured in place the same way. Indeed, except for interior features of the top section 203 configured to retain syringes, assembly 201 can be configured the same as assembly 1. It is noted, however, that for convenience, typically the top section 203 will be shallower than section 3, when the assembly 203 is configured to securing syringes, since there is not a need for as much depth in the section 203 by comparison to a section 3 configured to hold a dispensing pen (and replacement and used pen tips).

Referring, then, to FIG. 44, the dispensing assembly 212 includes a multi-chamber or compartment arrangement 213, each compartment 213c being provided with a cover 213x having an opening tab 213t and indicia 214 thereon, visually indicating days of the week and with touch or braille indicators. The particular dispenser 212 is a seven day dispenser, although alternatives are possible.

The cover section 203 is provided with an interior 203i configured with a medical dispenser containing holder arrangement 217, in this instance comprising a holder arrangement 218 for one or more syringes 219, and with a cover arrangement 220 for positioning over portions of the syringes 219, to secure the syringe(s) in place. (The syringes are not part of the assembly 201, and are depicted to show typical use).

Still referring to FIG. 44, typically the syringe(s) 219 would be “loaded” when initially stored within assembly 201. By “loaded” in this context, it is meant the syringes would be secured in place with material to be injected therein, so that the syringes 219 are ready for use. Of course, after the syringes 219 have been used, they could be stored “empty” or “used”, if not otherwise disposed of.

In FIG. 44, the syringes 219 are depicted stored “loaded” and thus ready for use. Each one of the syringes 219, then, includes a plunger 219p with a plunger tip 219t. In the example depicted, the plungers 219p are shown withdrawn, since the syringe chamber, not viewable under cover 220, is loaded with fluid. Each one of the syringes 219 is also depicted with a protective needle cover 219c thereon.

Still referring to FIG. 40, it is noted that, depending on the use and dosage, the syringes 219c may have varying amounts of liquid therein. The particular syringes 219 are viewed with the plungers 219p fully withdrawn, i.e., in a position corresponding to the syringes 219 being maximally full of injectable material. However, one or both of the syringes 219 may be prepared for a smaller injection, in which case the plunger 219p would not be as fully withdrawn.

It is important that the arrangement 210 securely hold the syringes 219c, so that the plungers 219p are properly secured in place against unintended movement. To provide for this, the holder arrangement 218 includes a plunger holder (fin) array 225 for each syringe 219; each array 225 comprising a plurality of spaced plunger holder fin members 225f.

Referring to FIG. 44, the fin members 225f of each array 225 are positioned on opposite sides of a receiver section or trough 225r, for the plunger 229p. Further, fins 225f comprise pairs of fins on opposite sides of the receiver 225r, that are spaced longitudinally along a length of receiver 225r, and are spaced sufficiently far apart from the next adjacent pair of the fins 225f to allow the plunger tip 229t to be received therebetween. Thus, the plunger tip 219t can be securely held in position along a variety of different selected locations, by the array 225.

Still referring to FIG. 44, it is noted that the holder 219 is configured to hold a plurality (in the example two) syringes 219. Of course alternate arrangements can be made using the techniques of the present disclosure, including ones in which the holder 219 is configured to only hold a single syringe, or others in which a different number are held.

Still referring to FIG. 44, attention is directed to cover 220. Cover 220 includes arcuate, longitudinal, syringe receiving troughs, receivers or sections 220r configured to snugly fit over and to receive a portion of a stored syringe 219. The cover 220 is pivotally secured in place along edge 220e by hinge 220h. Tab 220t is positioned for actuation of the cover 220.

From the above general characterization of features, operation and advantages of the assembly 201 can be understood. As with the assembly 1, assembly 201 comprises a container 202 that contains a multi-day or multi-chamber pill dispenser along with an alternate dispensing arrangement, in this example one or more syringes. The container 202 comprises a bottom section 204 and cover section 203, configured as a clamshell and openable along hinge 206. The bottom section 204 is configured to contain a multi-day pill chamber arrangement therein; the cover 203 being configured to contain an alternate dispenser 217 therein, in this instance a holder 217 configured to securely hold syringes 219. Advantageous features of the syringe holder 217 are characterized as including: cover/retainer 220; and, a fin array 225 for securely holding plunger tips 219t in place, with the array 225 configured for variable positioning of the plunger tips 219t.

In FIG. 45, assembly 201 is depicted in elevational view, open, and features referenced in connection with FIG. 44 are indicated by like reference numerals.

FIG. 46 is a cross-sectional view taken generally along line 46-46, FIG. 45. Individual portions of the assembly 201 characterized above, can be seen. Viewable in cross-section, are stem sections 219s of the plungers 219p for the individual syringes 219. Viewable in FIG. 47, is a cross-section of one of the chambers 213, showing cover 213x. It is noted that the construction of pill holder 212 and chambers 213 can be the same as described above for analogous assembly 1, see, for example FIGS. 4 and 5.

In FIG. 47, the closure arrangement 213a for an individual of the covers 213x is shown. The closure arrangement can be the same as described above for assembly 1.

In FIG. 48, an enlarged fragmentary cross-sectional view of an identified portion of FIG. 45 is shown. Here selected features of the cover 220 and the syringes 219 are shown. Hinge 220h can be seen as an optional living hinge between cover section 220s (with arches 220r therein) and a projection section 220p by which cover 220 is mounted to section 203. The mounting, discussed in further detail below, is by projection 220p extending into receiving groove 230 and cover section 203.

In FIG. 49, a projection/receiver arrangement is shown at 231 for securing the cover 220 closed. In FIG. 49, the arrangement 231 is shown not quite fully actuated. The cover 220 depicted with a flexible projection member 220q thereon, that is configured to engage, in a snap-fit manner, receiver 231r.

In FIG. 50, an exploded view of the assembly 201 is shown. But for the syringes 219, which are not part of assembly 201, the view of FIG. 50 is of the assembly 201 and its individual components. These components, to the extent previously identified, comprise: closure section 204, closure section 203, syringe holder 218 comprising cover 220 and a syringe rack or holder array 221 discussed below. Also viewable is multi-chamber pill holder 212, previously discussed.

In FIG. 50, hinge pin 207 is viewable, as well as latch actuator member 270.

The syringe rack or holder array 221, comprises: rack members 221p for securely holding one or more syringes; and, arrays 225 for securely holding plungers 219p. Further, attention is directed to receiving groove 230, configured to securely receive therein projection 220p on cover 220, during assembly.

Generally all portions of the assembly 201 depicted in FIG. 50 can be made from plastic can be readily assembled together, as discussed below, with a typical exception being hinge pin 207, which will typically be made from a metal such as a chrome steel. (The syringes 219, again, are not part of assembly 201, except in selected use).

In FIGS. 51-54, the bottom section 204 and its features are depicted. It is noted that the bottom section 204 can be identical to the bottom section 4/26 of the previously described embodiment. Thus section 204 is not discussed in detail here, but comprises analogous parts as follows: bottom cover 237; sidewall arrangement 238 with front and rear walls 239f, 239b respectively; hinge tubes 239t; end wall 238e with retainers 226p therein. The front wall 239f includes a recess 238w, for mounting of a latch actuator member. Front wall 238w also includes projections 238p and mounting pins 241. These identified parts have similar operations to these described for the previous embodiments.

In FIGS. 62-68, the multi-chamber medicine arrangement 212 is depicted in detail. It can be identical to multi-chamber arrangement 12, discussed in connection with the previous embodiment. As a result, its features are not discussed in detail herein, but are generally as follows. It comprises arrangement 328 including multiple covers 313x closing individual chambers 313. It includes mounting flange 360 and living hinges 313h. Again, it can be identical to the arrangement 12/28, with common features having the same function.

In FIGS. 55-61, features of the cover section 203 are viewable, independently of the syringes 219 and the cover 220. The features of the cover 203 depicted in FIGS. 55-61, then, comprise a component and features that can be molded as a single unit, from plastic.

It is noted that except for depth, the features of the cover 203 that define the top 203t and perimeter rim 203p can be the same as analogous features for the previously described embodiment. They are not described in detail herein, but generally comprise: front wall section 203f; rear wall section 203b; hinge tube sections 203t; and, latch arrangement projections 203y. Again, similar features have analogous function, to the previous embodiment.

Also viewable in FIG. 55 are selected features of the holder arrangement 218, comprised as previously discussed: fin array 225; syringe holder fin or rack 221p and groove 230.

In FIG. 56 a top plan view of cover 203 is viewable, in FIG. 57 and a cross-sectional view of FIGS. 58-60, a cross-sectional view is generally indicated. In FIG. 61, a view of an enlarged fragmentary portion of FIG. 60 is shown, depicting a recess 239 for helping cover 220 be retained locked closed.

In FIGS. 69-75, the cover 220 and its features are depicted. Referring to FIG. 69, cover 220 is viewable with cover section 220s having arch retainers 220r therein; tab 220t, hinge 220h and holder projection 220p. Also viewable is a flexible latch/projection member 350x, which can releaseably snap-fit engage recess 239, FIG. 56. The cover 220 of FIG. 69, is configured so that it can be molded as a single integral piece from a material such as an FDA approved random co-polymer polypropylene (pp).

In FIG. 70, member 220 is viewable in top plan view.

In FIG. 71, a cross-sectional view taken along line 71-71, FIG. 70 is shown.

In FIGS. 72 and 73, an enlarged fragmentary views of unidentified portions of FIG. 71 are shown.

In FIGS. 74-79, latch actuator member or panel 270 for the assembly 201 is shown. It can be an identical piece, operating in identical way, to member 31 discussed above for the previous embodiment, and is not further discussed here.

### IV. Observations Concerning Variations

### A. Holder Arrangement Variations

From the above discussions, it can be understood that a variety of holder arrangements can be used. Two holder arrangements depicted and described in the various embodiments, are a holder arrangement for a medical dispenser pen; and, a holder arrangement for one or more syringes. Still alternate holder arrangements can be used. For example the holder arrangement can be configured to hold a small bottle or vial of liquid, an inhaler or another medicine dispensing system.

In addition, a storage compartment arrangement is described for the embodiment of FIG. 1, for new pen tips and/or used pen tips. Storage compartments can be used in a variety of alternate systems, for other uses, along with other holders.

### B. Features Relating to Ease of Assembly

The features shown and described in the various embodiments depicted, are configured for convenient manufacture and assembly, from primarily plastic components (but for a preferred metal hinge pin). Alternate configurations can be used, if alternate assembly techniques and/or materials are accepted. However, the configurations are particularly advantageous for both assembly and operation, when provided with the features described and shown for plastic assembly.

### C. Alternate Closures

The closure arrangement can be varied substantially from that shown. The particular push panel arrangement depicted as convenient, as it can be easily managed by a variety of users. However, alternate latches such as slide latches and other engagement arrangements can be used.

### V. General Observations

According to the present disclosure, medicine dose containment arrangements features thereof and techniques for assembly and use of such arrangements, are described. In general, the medicine dose containment arrangements comprise a cover (second) section and a bottom (first) section. The first selected one of the cover section of the bottom section includes a plurality of individual pill containers therein. A second selected one of the cover section and bottom section includes a holder arrangement for medicine dispenser arrangement. Typically the holder arrangement is for a non-pill medicine dispenser arrangement, such as: a medicine dispenser pen; one or more medicine dispenser syringes; an inhaler; a small bottle containing liquid; etc.

In a typical assembly, the cover section is hingedly secured to the bottom section in a manner pivotable between closed and open orientations. Typically, the securement is with a hinge having a hinge pin, and the entire assembly is plastic but for the hinge pin, which would typically be made from metal such as chrome steel.

Typically the selected one of the cover section and the base section that includes the holder arrangement, is the cover section. In one example described, the holder arrangement comprises a holder for a medicine dispenser pen. When this is the case, the holder arrangement in an example depicted is optionally, a c-clip arrangement, for example an arrangement having two spaced flexible side c-clips.

In an example depicted, the selected one of the cover section and the base section includes at least one openable and recloseable equipment container therein. An example is provided in which the cover section includes multiple openable and recloseable equipment containers, for example to retain new pen tips and old pen tips.

In an example arrangement described herein, each openable and recloseable equipment container includes a hinged cover having a snap-fit closure and an opening tab.

In an example, each openable and recloseable equipment container comprises a sidewall structure including a mounting projection receiving slot; and, each equipment container includes a hinged cover having a mounting projection positioned projecting into the mounting projection receiving slot. An example arrangement, a snap-fit engagement is provided between mounting projection receiving slot and the mounting projection.

In a second embodiment described herein, the holder for the non-pill medicine dispenser arrangement comprises a syringe holder arrangement. An example syringe holder arrangement is provided that is configured for holding multiple syringes.

In a preferred embodiment depicted, the syringe holder arrangement includes a plunger tip stabilizing holder arrangement. The term “plunger tip stabilizing holder arrangement” and variants thereof, is meant to identify an arrangement that is configured to provide for secure holding of a plunger tip of a syringe, at a selected location, when a syringe is being secured by the assembly. An example described, this arrangement comprises at least a first array of spaced fins on opposite sides of the plunger receiver trough or space. The spaced fins are configured and located secure a plunger, of a stored syringe, in a selected one of various possible selected extension locations for the plunger. In this manner a stored syringe is securely held in a manner such that its plunger cannot move until the syringe is removed from the assembly.

In an arrangement depicted, the syringe holder arrangement comprises a syringe support rack and an openable and releasable hinged syringe cover. In that depicted example, the syringe holder comprises a syringe support rack capable of holding more than one syringe, portions of which are covered by the same syringe cover.

In an example depicted, the hinged syringe cover comprises at least one longitudinal syringe receiver arch. Each syringe receiver arch is an elongate extension or trough that receives a portion of a syringe snugly held therein, for secure engagement of the syringe.

In examples described, the assembly is configured as a clamshell holder, and a latch arrangement is provided to secure the clamshell closed until opening is intended. A closure latch actuator member is provided, to facilitate this. The closure latch actuator member, in examples depicted, comprises a pivotally mounted push panel member.

In an example arrangements depicted, multi-chamber pill container arrangement includes a plurality of independently covered pill chambers positioned in a single integral chamber containing member array. This integral chamber containing member array is then snap-fit as a single assembly, into a selected one of the cover section and the base section.

There is no specific requirement that an assembly component feature or technique that involve all of the detail described herein, in order to obtain some benefit according to the present disclosure.

What is claimed is:

1. A medicine dose containment arrangement comprising:
(a) a cover section and a bottom section;
(b) a first selected one of the cover section and the bottom section including a plurality of individual pill containers therein; and,
(c) a second selected one of the cover section and the bottom section including a holder arrangement for a medicine dispenser arrangement.

2. A medicine dose containment arrangement according to claim 1 wherein:
(a) the cover section is hingedly secured to the bottom section in a manner pivotable between closed and open orientations.

3. A medicine dose containment arrangement according to claim 1 wherein:
(a) the second selected one of the cover section and the bottom section is the cover section.

4. A medicine dose containment arrangement according to claim 3 wherein:
(a) the holder arrangement for a medicine dispenser arrangement comprises a holder for a non-pill medicine dispenser.

5. A medicine dose containment arrangement according claim 4 wherein:
(a) the holder arrangement for a medicine dispenser arrangement comprises a holder for a medicine dispenser pen.

6. A medicine dose containment arrangement according claim 4 wherein:
(a) the holder arrangement comprises a c-clip arrangement.

7. A medicine dose containment arrangement according to claim 6 wherein:
(a) the c-clip arrangement comprises two, spaced, flexible side c-clips.

8. A medicine dose containment arrangement according claim 1 wherein:
(a) the second selected one of the cover section and the bottom section includes at least one openable and recloseable equipment container therein.

9. A medicine dose containment arrangement according to claim 8 wherein:
(a) the second selected one of the cover section and the bottom section includes two openable and recloseable equipment containers therein.

10. A medicine dose containment arrangement according to claim 8 wherein:
(a) each openable and recloseable equipment container includes a hinged cover having a snap-fit closure and an opening tab.

11. A medicine dose containment arrangement according to claim 8 wherein:
(a) each openable and recloseable equipment container comprises a sidewall structure including a mounting projection receiving slot; and
(b) each openable and recloseable equipment container comprises a hinged cover having a mounting projection positioned projecting into the mounting projection receiving slot.

12. A medicine dose containment arrangement according to claim 4 wherein:
(a) the holder for a non-pill medicine dispenser comprises a syringe holder arrangement.

13. A medicine dose containment arrangement according to claim 12 wherein:
(a) the syringe holder arrangement includes a plunger tip stabilizing holder arrangement.

14. A medicine dose containment arrangement according to claim 13 wherein:
(a) the plunger tip stabilizing holder arrangement comprises at least a first array of spaced fins on opposite sides of a plunger receiver trough;
(i) the first array of spaced fins being configured to secure a plunger tip, of a syringe, at a selected one of various possible selected extension locations.

15. A medicine dose containment arrangement according to claim 12 wherein:
(a) the syringe holder arrangement is configured to secure a plurality of syringes.

16. A medicine dose containment arrangement according to claim 12 wherein:
(a) the syringe holder arrangement comprises a syringe support rack and an openable, hinged, syringe cover.

17. A medicine dose containment arrangement according to claim 16 wherein:
(a) the hinged syringe cover comprises at least one longitudinal syringe receiver arch.

18. A medicine dose containment arrangement according to claim 1 wherein:
(a) the first selected one of the cover section and bottom section includes a container closure latch actuator member wherein:
(i) the closure latch actuator member comprising a pivotally mounted push panel actuator member.

19. A medicine dose containment arrangement according to claim 1 containing:
(a) a multi-chamber pill container arrangement comprising a plurality of independently covered pill chambers positioned in a single integral chamber containing member:
(i) the single integral chamber containing member being snap-fit secured in the first selected one of the cover section and the base section.

20. A medicine dose containment arrangement according to claim 1 wherein:
(a) the bottom section and cover section are secured to one another with a hinge including a hinge pin; and,
(b) the arrangement is entirely molded plastic except for the hinge pin.
